(19) United States
(12) Patent Application Publication
(10) Pub. No.: US 2021/0113753 A1
(43) Pub. Date: Apr. 22, 2021

(54) DIALYSATE-FREE WEARABLE RENAL REPLACEMENT SYSTEM

(71) Applicant: University of Florida Research Foundation, Inc., Gainesville, FL (US)

(72) Inventor: Saeed Moghaddam, Gainesville, FL (US)

(21) Appl. No.: 17/072,983

(22) Filed: Oct. 16, 2020

Related U.S. Application Data
(60) Provisional application No. 62/916,636, filed on Oct. 17, 2019.
(60) Provisional application No. 62/926,982, filed on Oct. 28, 2019.

(51) Int. Cl. A61M 1/16; A61F 5/445; B01D 71/02; B01D 61/46; B01D 61/02; B01D 71/56; B01D 61/58

(52) U.S. Cl. CPC A61M 1/16; A61F 5/445; B01D 71/024; A61M 2202/0498; B01D 61/025; B01D 71/56; B01D 61/58; B01D 61/46

(57) ABSTRACT

Various examples are provided related to dialysate-free renal replacement. In one example, a dialysate-free continuous renal replacement system includes a blood filtration stage (e.g., in a microfluidic membrane module). The blood filtration stage can include a blood filtration membrane configured to that can provide a filtered fluid by renal filtration of blood passing through the blood filtration stage at arterial pressure. The continuous renal replacement system can also include a salt recovery stage and a water recovery stage. The salt recovery stage can recover ions through separation from the blood filtration stage. The water recovery stage can separate water from the desalted fluid from the salt recovery stage, where the water is combined with the separated ions and reinfused into the blood after passing through the blood filtration stage.

### CROSS REFERENCE TO RELATED APPLICATIONS

This application claims priority to, and the benefit of, co-pending U.S. provisional application entitled “Dialysate-Free Wearable Renal Replacement System” having Ser. No. 62/916,636, filed Oct. 17, 2019, and co-pending U.S. provisional application entitled “Dialysate-Free Wearable Renal Replacement System” having Ser. No. 62/926,982, filed Oct. 28, 2019, both of which are hereby incorporated by reference in their entireties.

### BACKGROUND

The end-stage renal disease (ESRD) is a chronic disease that exerts a great negative impact on patient's quality of life mainly due to the accompanied impairment and imposed limitations on almost all domains of their daily lives. The number of patients with ESRD is progressively increasing and the demand for renal replacement therapies (RRTs) is expanding; with diabetes and high blood pressure being the two leading causes of ESRD. The standard care for these patients is lifelong hemodialysis (HD) or hemodiafiltration (HDF) treatments thrice-weekly. This in-center thrice-weekly treatment has patient related problems, is expensive, and has poor outcomes such as increased risk of cardiovascular events and mortality due to the extra-long interdialytic period. Post dialysis recovery time can be twice as long as the treatment time, during which patients report feeling ill, rundown, and depressed; this prevents most patients from holding a full-time job.

Diseases associated with ESRD are many; with hypo- or hypertension, abdominal and muscle cramps, nausea, shortness of breath, itching, anemia, sleep problems, bone loss, cardiovascular, amyloidosis (pain due to deposition of proteins on joints and tendons) and depression being common. Life expectancies for ESRD patients have improved little in the past two decades, particularly for those 50 years of age and older. ESRD treatment accounts for 7% of all Medicare spending ($31B) and places an extremely high financial burden on the medical system. While it is considered a routine therapy in prosperous nations, it is not accessible or unaffordable in some areas of the planet. The technology behind this treatment has been slow to evolve over the last few decades, limiting the opportunity to make significant improvements in patients' lives in US and abroad. To address patients' safety concerns and enhance affordability in US and throughout the world, there is a need for membrane improvements that facilitate toxin removal at low operating flow rates.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of transforming the treatment of chronic kidney disease in patients, in accordance with various embodiments of the present disclosure.

FIG. 2 is a cross-sectional schematic diagram illustrating an example of a glomerular basement membrane, in accordance with various embodiments of the present disclosure.

FIG. 3 illustrates a comparison of small molecule diffusive permeability in an ultrathin silicon nanomembrane to conventional dialyzer membranes, in accordance with various embodiments of the present disclosure.

FIG. 4 is an illustrative depiction of a graphene oxide (GO) bilayer membrane, and species transport path, in accordance with various embodiments of the present disclosure.

FIG. 5 is a schematic diagram illustrating flows of a three-stage system for continuous renal replacement therapy (CRRT), in accordance with various embodiments of the present disclosure.

FIG. 6 illustrates an example of a microchannel design and system for sieving characterization, in accordance with various embodiments of the present disclosure.

FIGS. 7A-7F illustrate examples of activity and characterization of GO hemocompatibility tests, in accordance with various embodiments of the present disclosure.

FIGS. 8A-8D illustrate an example of an electrodialysis desalting system for testing and results, in accordance with various embodiments of the present disclosure.

FIGS. 9A and 9B illustrate an example of a water recovery and waste removal system for testing and results, in accordance with various embodiments of the present disclosure.

### DETAILED DESCRIPTION

Disclosed herein are various examples related to dialysate-free renal replacement. ESRD patients frequently have azotemia (abnormally high levels of nitrogen-containing compounds such as urea, creatinine, various body waste and other nitrogen-rich compounds) and fluid overload. ESRD patients require frequent hospital or dialysis centers visits, mainly thrice-weekly for a 3-4 hours long procedure, thus requiring substantial changes in their normal way of life. Post dialysis recovery time can be twice as long as the treatment time, during which patients report feeling ill and rundown.

The most common form of care is a process during which the patient is connected to a machine that draws the patient's blood, most commonly, through an arteriovenous (AV) fistula (a surgically created connection between an artery and a vein). A pump draws patient's blood at a rate of 300-400 mL/min and returns it to a vein that has not evolved for such high blood infusion rate, which can potentially lead to its ballooning (aneurysm) and potential rupture over time. This rapid withdrawal of blood results in blood pressure changes and can cause loss of consciousness. Over the duration of this procedure, 60-70 L (about the interstitial liquid volume of an adult person) of blood is processed through the machine while species from body's interstitial liquid diffuse into the bloodstream. At the end of each procedure, about 100-150 mL of blood is lost to a bulky membrane module and the extracorporeal blood circuit. At the end of each procedure, 100-150 mL of blood can be lost to a bulky membrane module and the extracorporeal blood circuit.

Those who are managed with intermittent thrice-weekly treatment need fluid restriction, and the ability to meet their higher amino acid needs to compensate for hypercatabolism is often hindered by accumulation of nitrogenous waste. Hence patients can greatly benefit from more frequent intermittent treatment to achieve metabolic and azotemic control. More frequent intermittent treatment allows more fluid intake depending on the patient's fluid status and tolerance. For hemodynamically unstable patients, frequent treatment (and ideally continuous renal replacement therapy, CRRT) is the preferred choice because it allows for slow continuous fluid removal and superior hemodynamic and metabolic control. More frequent low flow rate renal replacement therapies (RRTs) that can be used in the comfort of home, potentially overnight, or as a wearable RRT can dramatically change ESRD patients' lives and reduce societal costs. Patients can experience cardiovascular improvements and normotensive blood pressure as well as better sleep, anorexia, and cognitive functions.

While significant effort has been made to introduce a portable or wearable artificial kidney (WAK), there have been setbacks and no commercial system is currently available. The fundamental challenge is the operation principle of the systems and transport characteristics of the membranes utilized in these systems, which have remained unchanged despite decades of engineering. Wearable RRT have been constrained by the engineering challenge of replacing the traditionally very large volumes of dialysate with a system that can be carried. The use of sorbent canisters can regenerate small volumes of dialysate fluid. However, this approach is limiting because the canister must be regularly recharged or replaced, preventing a truly CRRT. Reference will now be made in detail to the description of the embodiments as illustrated in the drawings, wherein like reference numbers indicate like parts throughout the several views.

Nanoengineering of miniature systems offers the ability to provide lifestyle benefits in the form of mobility and convenience and treatment outcomes by enabling more frequent or continuous dialysis (keeping uremic toxin levels steady and maintaining consistent water balance) and minimizing extracorporeal blood circuit volumes. Advancements in nano science and engineering provide an opportunity to vastly improve the dialysis technology and also develop an alternative to the dialysis process that can recapitulate kidney's function, producing urine without external fluids and absorbents at, e.g., two orders of magnitude smaller size than current “kidney in a backpack” systems—a truly wearable kidney. A graphene oxide membrane has been developed that can be 40× and 5× more permeable than the commercial and kidney's glomerulus membranes, respectively. This can enable, e.g., a 5×5 cm² footprint membrane module that can be directly connected to vascular access eliminating the extracorporeal blood circuit and pump alleviating the fear of exsanguination, a key barrier to adoption of in-home dialysis. Due to the far superior fouling performance of the GO membrane relative to the existing polymer membranes, the device may only need to be checked/replaced in a clinic weekly/monthly.

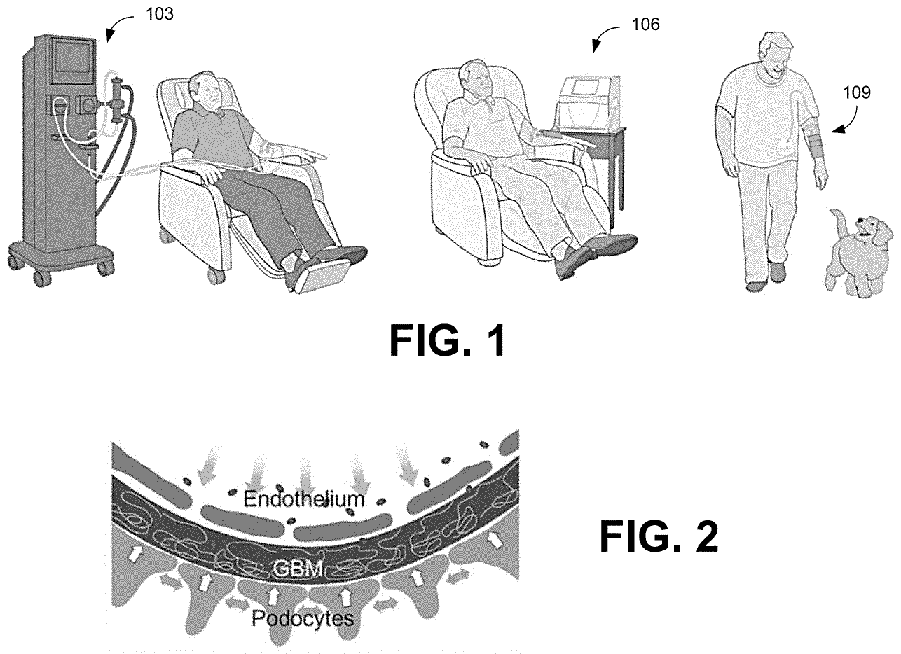

FIG. 1 illustrates an example of the progression of development from the current treatment 103 for chronic kidney disease to a more reliable home-based treatment enabled by miniaturized graphene oxide (GO) membrane module that allow operation with arterial pressure 106 and a wearable dialysate-free system 109. Adoption of these technologies can dramatically improve patient's health and quality of life. Engineered GO bilayer membranes can enable a paradigm shift in the treatment of chronic kidney disease patients. Highly permeable nanomembranes can be engineered to miniaturize the membrane module. Also, a novel three-stage filtration system that can recapitulate many of the separation, transport and re-absorptive properties of the kidney, can eliminate the need for sorbents or large volumes of dialysate.

The miniaturized GO membrane module 106 can benefit from a microfluidic membrane module, enabled by an ultra-high-throughput and selective nanoengineered membrane, operating at a low flow rate of, e.g. 100 mL/min or less. The low pressure drop (e.g., 3 kPa or less) of this device can allow operation using arterial pressure which could facilitate elimination of the blood pump and associated blood tubing. Depending on its membrane design, it can be utilized in both hemodialysis (HD) and hemodiafiltration (HDF) configurations. This miniaturized GO membrane module 106 can enhance adoption of homebased RRT. While advancements have been made in homebased RRT, the actual adoption rate is low because systems are similar to those in the clinic which require blood flow rates of 300-400 mL/min raising fear due to the risk of bleed out.

A full-scale parallel plate tangential flow (TF) membrane module can be developed for 1) home-based frequent intermittent dialysis, 2) potential home-based hemodiafiltration (HDF), and/or 3) first stage of a three-stage wearable dialysate-free continuous renal replacement therapy (CRRT). A multilayer microchannel device with a footprint of about 5×5 cm² and <1 cm in thickness can be adequate for treatment of an adult patient, and can be installed on the patient's forearm.

The use of dialysate fluid hinders full mobility. The wearable dialysate-free system 109 can eliminate the need for 100-150 L of dialysate fluid in dialysis treatment modality and 10-25 L of infusion solution needed in HDF. Here, the infused solution is produced internally within the system by reprocessing the filtered plasma. This action minimizes and/or eliminates problems of sterility and apyrogenicity known to cause inflammatory responses, particularly in elderly and patients with diabetes and higher risk of cardiovascular problems. The system can provide online nubs for correction of electrolytes, blood water volume, as well as acid-base balance.

A full-scale dialysate-free wearable system may be implemented based on a three-stage system that can sufficiently perform the filtration function of a kidney without dialysate fluid. A model of this system has been prepared and the overall viability of each stage examined separately in a set of feasibility tests at small scales and limited species. The first stage of the system can process <100 ml/min blood flow rate. Using arterial pressure, the membrane can allow this stage to filter out <10 ml/min blood plasma into the 2nd stage. A 2nd stage membrane-based electrodialyzer (ED) can recover the filtered plasma salt and direct a solution containing mostly urea and middle-weight toxins into a third stage, where water can be recovered using a reverse osmosis (RO) membrane cleansing process while concentrated urine is produced and discharged into a urostomy bag at a rate of 1-2 mL/min, similar to the rate removed by normal kidneys. In addition to urea and toxin clearance, the blood pressure can be regulated by automatically adjusting the water volume it returns to the vein. The system can also meter the rate of salt reinfusion back into blood flow potentially liberating patients from challenges associated with increased salt concentrations.

Nanoengineered High Throughput and Highly Selective Membranes

Membranes are the fundamental building block of the hemofiltration process. Their permeability determines size of the membrane module and their selectivity impacts the system design configuration. Membranes can mimic the permselectivity of the glomerular filtration barrier (GFB) of the kidney. GFB is composed of a glomerular basement membrane (GBM) that can be about 350-nm-thick, lined by fenestrated endothelium (with 50-100 nm diameter pores) on the blood side and a specialized epithelium (named “podocytes”) on the urinary space side. FIG. 2 is a cross-sectional schematic diagram illustrating GBM. GBM and the glomerular epithelial slit diaphragms (GESD) are responsible for permselectivity and maintaining a highly regulated barrier that allows passage of water, small solutes/ions, and smaller proteins transport but not plasma proteins larger than about 65 kDa. Existing synthetic membranes, such as Fresenius Polysulfone High-Flux membrane (max. pore size about 5 nm), allow permeation of up to 60-70 kDa species. However, these membranes are an order of magnitude thicker (about 10 μm) than the GBM.

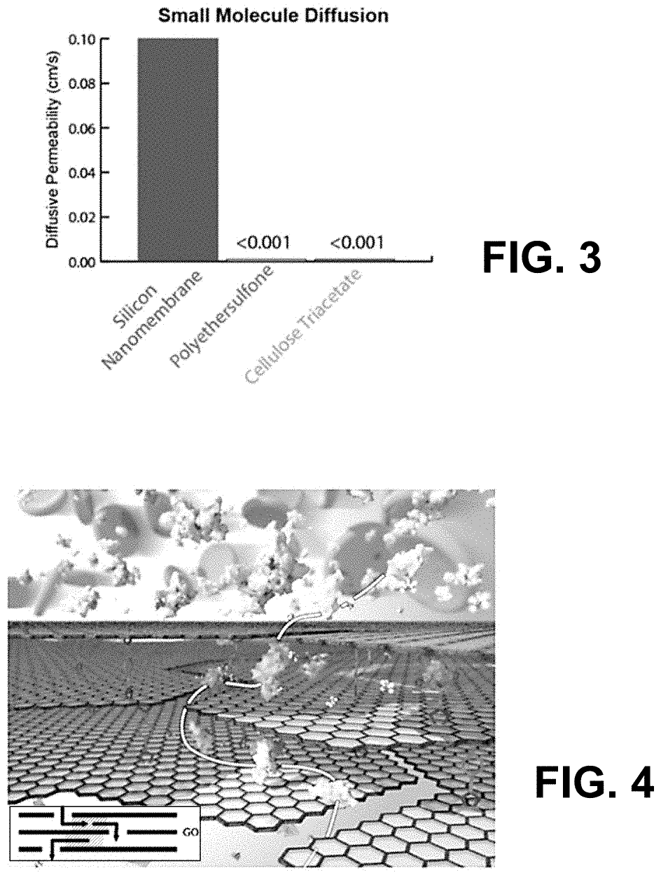

Nanoengineering allows fabrication of membranes that are an order of magnitude thinner than the GBM. FIG. 3 illustrates the transformative impact of such a membrane in this field. Ultrathin membranes can enable efficient hemodialysis. A silicon nitride (SiN) membrane can include two orders of magnitude higher diffusive permeability relative to existing synthetic membranes. FIG. 3 illustrates an example of small molecule diffusive permeability in ultrathin silicon nanomembranes compared to conventional dialyzer membranes. A microchip utilizing this technology has been used to treat a uremic rat model, and results showed near normal urea concentration reached with the SiN microchip dialyzer while the urea clearance rate achieved with commercial membranes polyethersulfone (PES) and cellulose triacetate (CT) was less than the metabolic generate rate, leading to a slow rise in the urea concentration.

GO-based membranes can be used as an alternative to SiN in this application. Analysis of the impact of GO physicochemical properties on transport through GO laminates suggested that unparalleled permeability and selectivity can be achieved with GO-based membranes. Separation of a few endocrine disrupting compounds (EDCs) (e.g., ibuprofen) has been carried out, and the membrane permeability can be enhanced by two orders of magnitude relative to the tested membrane. Successful verification of the proposed hypothesis and implications are provided later in this document. FIG. 4 shows a depiction of a GO layer membrane, with the inset illustrating the species transport path. First, the GO bilayers membrane configuration and its unique properties are briefly explained.

GO is an atomically-thin functionalized derivative of graphene, comprising a carbon backbone with several oxygen-containing groups (e.g., epoxy, hydroxyl, carboxyl, carbonyl) on the basal plane and edges. Due to its functional groups, the GO surface can be extensively modified with numerous molecules. Parameters such as surface charge polarity and density and hydrophilic and hydrophobic characteristics of GO surface can be changed through grafting and molecular self-assembly, enabling mimicking surface properties of glomerular barrier surface known to be negatively charged (by sialoglycoproteins, peptidoglycans, etc.) to restrict filtration of negatively charged macromolecules (e.g. albumin) relative to neutral ones. Additional information regarding the synthesis and fabrication of the PMMA-PAH-GO laminate structure can be found in “Bilateral 2D material laminates for highly selective and ultra-high throughput filtration” (U.S. patent application Ser. No. 16/382,851 filed Apr. 12, 2019), which is hereby incorporated by reference in its entirety.

Another unique transport characteristic of an assembly of GO sheets (platelets) is that, unlike other membranes that have a range of pore size, a bottleneck (e.g., shaded in FIG. 4 inset) formed between GO platelets sets a precise molecular size cut-off. The effective sieve size of a GO laminate was found it to be about 9 Å under aqueous conditions. This interlayer spacing can be readily adjusted using different size interlinking molecules. Experimental and theoretical studies suggest that a microchip with a footprint of about 5×5 cm² comprising about 15 stacked microchannel layers (<1-cm-thick) is adequate for performing HD and HDF at low flow rates. Existing machines operate at a flow rate of about 400 mL/min over a total duration of about 9 hours/week. Hence, a device flow rate of about 50-75 mL/min would likely be sufficient for nightly dialysis sessions.

Dialysate-Free Wearable Continuous Renal Replacement Therapy

Manmade blood cleansing processes are fundamentally different than the renal filtration process. In a kidney, nephron's tubular reabsorption mechanisms return most of the water and solutes into extracellular fluid and blood circulatory system. In HD process, dialysate which interfaces the blood from across the membrane must be balanced electrolytically to prevent rapid desalting of blood. And, in an HDF process that involves convective transport across the membrane (similar to glomerular filtration), 10-25 L of electrolyte is infused back into the bloodstream in each session to make up for the filtered salt solution. The amount of dialysate for HD or dialysate and reinfusion fluid for HDF (many liters) does not liberate ESRD patients from confinement of a chair/bed during treatment.

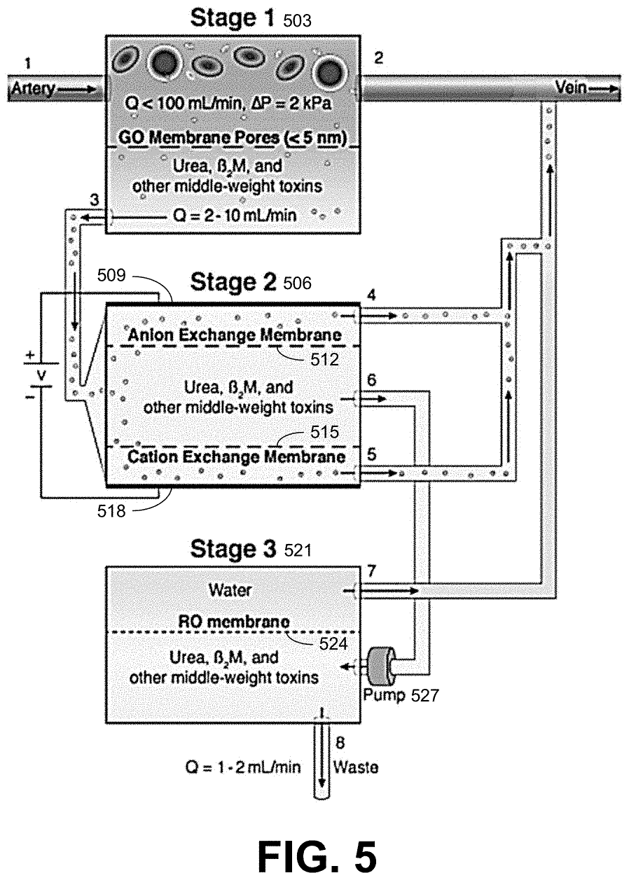

To eliminate the need for both dialysate and reinfusion fluid, a system comprising stages for the recovery of the filtrate salt and water can be used. FIG. 5 shows a diagram illustrating flow in an example of a three stage system based on this approach. Blood flows through (blood filtration) stage 1 (503) from an artery (stream 1) and is returned to a vein (stream 2).

Stage 1 (503) functions similar to a glomerular filtration, and an HDF membrane module. Ions passing through the GO membrane of stage 1 (503) are passed (stream 3) to (salt recovery) stage 2 (506) where they can be recovered using an electrodialysis (ED) process. This ED process is particularly efficient in salt concentrations that are relevant here. In the ED process, negative ions migrate towards the positive electrode (anode) 509, pass through an anion exchange membrane (AEM) 512, and enter the anode compartment. Positive ions (cations) within this channel while repelled by the positive electrode 509 cannot exit the anode compartment because AEM 512 is impermeable to cations. Eventually, these negative ions along with protons (H+) generated at the anode 509 exit the anode compartment (stream 4). Similarly, positive ions migrate towards negative electrode (cathode) 515, pass through the cation exchange membrane (CEM) 518, and ultimately exit cathode compartment (stream 5) along with negative ions (anions) within that channel (CEM 518 prevents their exit from cathode compartment) and hydroxyls (OH—) produced at the cathode 515. In alternative embodiments, the polarity of the electrodes can be changed to recover the salt in the middle compartment of stage 2, rather than within the electrodes' compartments. In this case, the ion-depleted streams (in the electrodes' compartments) can be directed to the third stage.

Desalted fluid containing urea and middle-weight toxins in the middle compartment of stage 2 flows out (stream 6) to (water recovery) stage 3 (521) where it is cleansed by a reverse osmosis (RO) membrane 524. The RO membrane 524 can utilize a polyamide layer that allows water to diffuse through while rejecting other species (advance HD and HDF machines use a similar membrane to clean water for dialysate and reinfusion fluids). Streams 4 and 5 are mixed with water returning (stream 7) from stage 3 (521) and reinfused back into the bloodstream (along with a fraction of urea, creatinine and toxins). Ultimately, urine at a rate of about 1-2 mL/min is discharged to a urostomy bag (stream 8).

In this dialysate-free system, stages 2 and 3 (506/521) implement ion and water recovery processes similar to the nephron's loop of Henle. The ascending branch of loop of Henle pumps salts out of the filtrate against their gradient using ion pumps energized by ATP (adenosine triphosphate). In the system of FIG. 5, energy to move ions against their gradient is provided by the electric field. In the descending branch of the loop of Henle, water is reabsorbed through osmotic pressure (via water selective aquaporin) established between the tubule and salty Medulla (which receives the actively pumped salt). In FIG. 5, external pumping force (e.g., pump 527) can be utilized to pass water against its gradient through the water selective RO membrane 524.

At a filtrate rate of 10 mL/min in stage 1 (503), under continuous operation, the system can provide a total reinfusion of about 80-90 L that is equivalent to a total reinfusion in 3 high-rate HDF sessions. In addition to the benefits associated with a slow and continuous system discussed above, the capability of the proposed CRRT to remove fluid steadily from the vascular space at a volume similar to that physiologically removed by normal kidneys gives the treating physician the ability to keep the patients euvolemic, regardless of the amount of fluid they may ingest. Furthermore, the elimination of excess fluid may result in better control of hypertension. In addition, the salt content of the reinfused filtrate (exit streams 4 and 5 of stage 2) can be actively controlled (by adjusting the applied voltage to allow salt discharge with stream 6 and which is ultimately filtered out into the waste stream 8 in stage 3) to liberate salt intake for ESRD patients. This system can also allow reinfusion of streams 4 and 5 at different rates (the balance can be directed to waste stream 8) to serve as the acid-base balancing function of the kidney.

Stage 1 Membrane Transport Characteristics

Relations between GO nanoplatelets interlayer spacing and sieving rates of ions and middle weight solutes can be developed. For example, the ion rejection rate declines (i.e., more ions pass through the membrane) as the interlayer spacing is increased. Membranes with interlayer spacings of 3-5 nm can provide transport characteristics which are beneficial to the design of the stage 2 membrane module (FIG. 5). FIG. 6 is a schematic diagram illustrating an example of channels (top view) disposed on opposite sides of a membrane (e.g., a GO membrane) that may be used for blood filtration in stage 1.

Measurements of GO laminates ion rejection capability at seawater salt concentration of 3.5% (4 times higher than physiological concentration of about 0.9%) showed rejection rates of >99.5% for Na⁺, NH₄⁺, K⁺, Mg²⁺, Ca²⁺, F⁻, Cl⁻, NO³⁻, and/or SO₄²⁻ at an interlayer spacing of 1 nm. To allow clearance of middle weight solutes such as B2M, free leptin (16 kDa) (a uremic toxin implicated in malnutrition and anorexia), cytokine, proinflammatory monocytes and acute phase proteins, oxidized-derived products, and free paracresol or paracresyl sulfate or indoxyl sulfate to levels achieved in HDF, interlayer spacing must be increased well beyond 1 nm.

GO layers with 2-3 nm separation can provide significant ion rejection due to overlapping (about 1 nm) Debye layers in physiological conditions. Further increase in spacing to 4-5 nm could lead to substantial decline in rejection. Imposing a minimum clearance of 13.5-18 grams of salt can maintain a normal salt concentration. The upper limit of salt clearance can be defined based on salt recovery efficiency of stage 2 (FIG. 5). Sieving performance of three membranes with interlayer spacings of 3-5 nm can be measured at representative average blood salt concentrations; Na⁺ (140 mEq/L), K⁺ (4 mEq/L), Cl⁻ (100 mEq/L), HCO³⁻ (24 mEq/L), Mg²⁺ (2 mEq/L), Ca²⁺ (2.5 mEq/L), PO₄³⁻ (1 mEq/L) and/or SO₄²⁻ (0.5 mEq/L). Sieving rates of four uremic toxins B2M (11.8 kDa), interleukin-18 (18 kDa), adiponectin (30 kDa), and pentraxin-3 (40 kDa) can be measured as representative of 18 uremic toxins for which there is evidence of a link to inflammation and/or cardiovascular disease. These studies can provide a library of data that can be used to form decisions about the design of stage 2.

The efficacy of a membrane was demonstrated utilizing three GO layers. To achieve a high selectivity with only three GO layers, a near-atomically smooth underlaying substrate was used. Three polymethyl methacrylate (PMMA) membranes with pore sizes of approximately 100, 200, and 400 nm were nanoimprinted and then hydrolyzed for the GO nanoplatelet sizes. In order to determine the membrane transport properties, microfluidic test devices as shown in FIG. 6 were used.

A set of tests were conducted to determine the membrane permeability and selectivity of different species. First, water permeability of the membrane was measured. Reducing the nanoplatelets size directly improved the permeability, through shortening the effective transport path length. A permeability of 1562 mL/hr·mmHg·m² was measured, two orders of magnitude higher than existing nanofiltration membranes. The permeability of the membrane is nearly fortyfold higher than the commercial high flux hemodialysis membranes (e.g., EVODIAL 2.2 and ELISIO-9H manufactured by Baxter Healthcare Ltd. and Nipro Medical Corp., respectively). Such a membrane offers value to the hemodialysis industry. Additionally, the GO membrane permeability is nearly 5 times greater than that of the glomerular membrane of the kidney, demonstrating the unique capability of nanomaterials to exceed their biological equivalent. Another unique transport characteristic of the membrane is its precise MWCO that offers ultimate selectivity relative to polymer membranes that have a range of pore sizes.

With such a high permeability, a fraction of a square meter can be used for dialysis of a human adult. Such a small membrane area can be built into a small multilayer microfluidic cartridge to reduce the extracorporeal blood circuit by an order of magnitude, preventing loss of 100-150 mL blood in each dialysis session. The membrane module can be operated with hemodynamic pressure rather than an external pump that is a source of hemolysis due to the high internal shear forces. This can enhance quality of care while reducing costs, particularly through increasing home dialysis, with negligible contribution from the membrane module itself.

A second set of tests were conducted to determine the transport rate and selectivity of different spices. The membrane sieving capability of urea and cytochrome-c as representative small and middle-weight uremic toxins, while monitoring the retention of albumin, were examined. A maximum urea sieving coefficient of 0.5 was achieved while the human serum albumin (HSA), with a size of <66 kDa, retention was >99%. In order to accommodate a nocturnal dialysis flow rate on the order of 100 mL/min, it is anticipated that an effective sieving area as little as 0.015 m² would be sufficient, considering a membrane surface area of 2.5 mm² used in the 0.017 mL/min test device. This membrane area can be incorporated in a microfluidic membrane module with a 5×5 cm² footprint with 15 microchannel layers.

The hemocompatibility of the GO-based membranes where compared to commercially available hemodialyzer materials. The GO-blood interactions were investigated, utilizing careful control on oxidation extent and GO nanoplatelet size to identify their hemocompatibility. GO was synthesized, sonicated in a bath sonicator, and then deposited onto glass substrates, where complete coverage of the glass substrate with GO nanoplatelets prevents the substrate contribution toward hemolysis. SEM imaging showed close packing of GO nanoplatelets on the glass substrate with minimal exposure of the underlying glass, where layers of GO-PAH fully covered the surface.

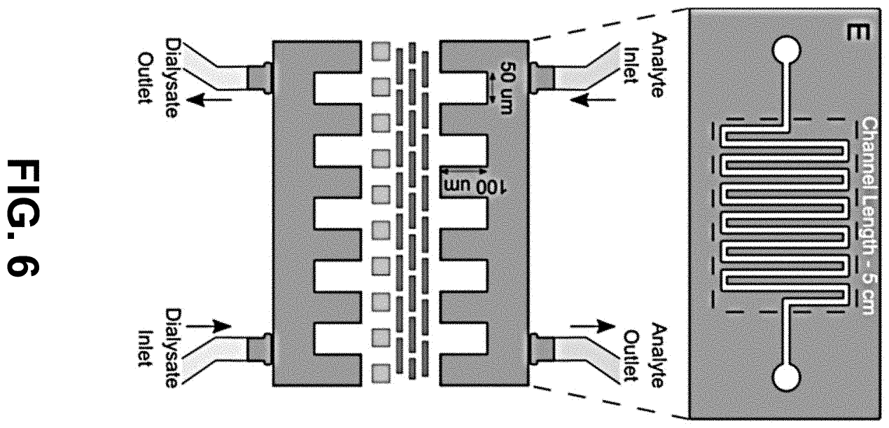

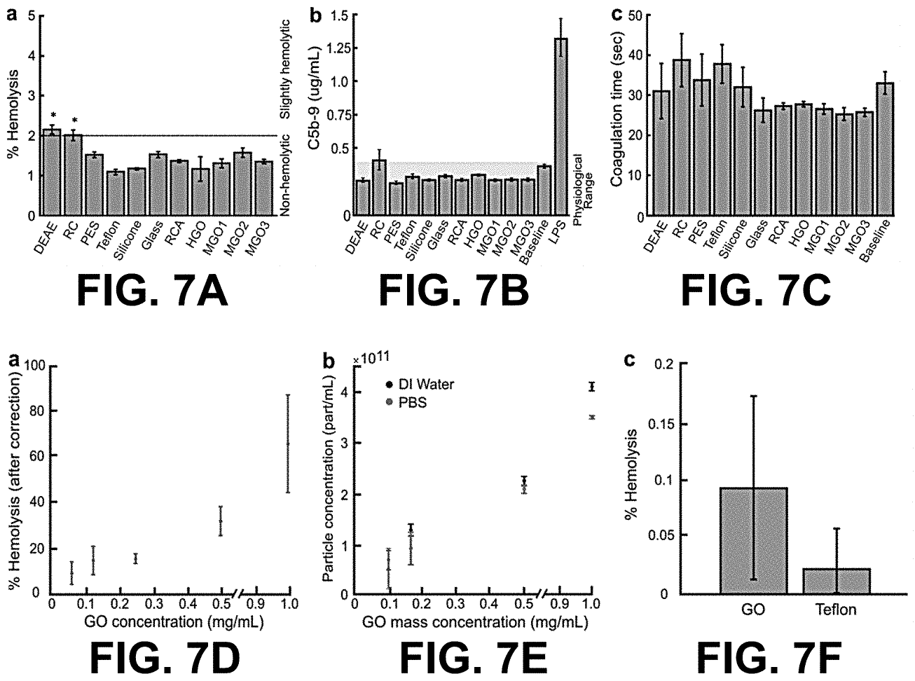

Hemolysis, the rupture of red blood cells, was investigated following 1 hour of exposure to the membrane surfaces. It was found that diethylaminoethyl cellulose (DEAE) and regenerated cellulose (RC) membranes, which fell out of favor as hemodialysis materials, induced a slight level of hemolysis (˜2%), as expected. These membranes produced notably higher hemolysis compared to the tested GO oxidation assemblies. FIGS. 7A-7C show examples of the hemolysis results. FIG. 7A shows hemolysis results for the GO membrane conformation with varying oxidation factors and comparative commercial substrates. FIG. 7B shows complement activation results for the GO membrane layout. Each bar and the associated error bar represent the mean and the standard error of six independent samples (n=6). For positive control (LPS) and the negative controls at 4° C. and 37° C., n=3. FIG. 7C shows coagulation results for GO and standard membrane material. Teflon, silicone, and glass, which were used in the GO testing apparatus, showed comparable hemolysis levels to the different GO substrates indicating that the actual GO contribution to hemolysis may be even lower than that measured.

Substrate-induced immunogenicity, as assessed by the production of C5b-9 complement, was then investigated. Thrombogenicity, or the tendency of a material to induce clotting, of the GO surface was evaluated based on coagulation time after post-thrombin addition, where shorter times for coagulation onset corresponded with higher thrombogenicity. No statistically significant differences were observed across all GO variants compared to the control substances. Compared to polyethersulfone (PES) membranes and Teflon, noted for their highly hemocompatible characteristics, GO membranes exhibit no significant variance in hemolytic, coagulation, or complement activation characteristics as shown in FIGS. 7A-7C. There was no statistical significance between GO substrates and PES membranes.

These hemocompatibility results are contradictory to prior GO compatibility studies, which indicated that GO induces high levels of hemolysis and complement activation. Comparison between GO suspension and membrane platforms suggest that the interactions between red blood cells (RBCs) and GO platelets in these two cases fundamentally differ. The ability of GO nanoplatelets in suspension to freely diffuse leads to an increased interaction rate with other species. FIGS. 7D-7F illustrate characterization of GO suspension behavior and quantification of hemolytic behavior after perfusion at physiological conditions. FIG. 7D illustrates the GO suspension hemolysis using GO 60-minute sonication at varied concentrations. FIG. 7E illustrates GO aggregation in DI water and PBS solutions based on the number of particles present with higher aggregation in PBS. FIG. 7F illustrates hemolysis observed after perfusion across GO surfaces, which fall in the non-hemolytic regime (<2%).

A nanoplatelet size distribution ranging from 150-500 nm was analyzed through nanoparticle tracking analysis (NTA). When freely diffusing, a randomly oriented GO nanoplatelet with an estimated disc diameter of 150 nm has a diffusion coefficient of about 2.9 μm²/sec. At an RBC concentration of 5×10⁸/mL and a GO concentration of 3.5×10¹⁰/mL, this translates to a GO-RBC encounter frequency of about 82 times every 20 sec. This interaction frequency is drastically higher compared to the bilayer scenario, as RBC sedimentation tends to occur, limiting the number of RBCs which can actively interact with the surface. Even when accounting for recirculation of the RBCs atop a GO laminate at 10 dyn/cm² that might occur in a wearable hemodialyzer, the hemolytic activity is comparable to polymer baselines and well below GO suspensions as shown in FIG. 7F. These results suggest that the primary hemolytic mechanism found in suspension studies is absent in GO laminates or occurs on a much less pronounced magnitude.

A unique self-assembled GO nanoplatelet ordered mosaic has been demonstrated, advancing the development of graphene-based membranes. The membrane included three layers of GO atop a PMMA support, achieving permeabilities as high as 1562±30 mL/m2·hr·mmHg, nearly two orders of magnitude greater than existing nanofiltration membranes. A precise effective pore size of 5 nm offers a great advantage over the polymer membranes with a range of pore sizes. This GO laminate has also shown vastly improved hemolytic and biocompatible properties compared to previous studies concerning GO nanoplatelets in suspension. Even under recirculation conditions of 10 dyn/cm², hemolytic activity of GO laminates remains at or below the commercially available dialyzers. The membrane provides a viable platform for miniaturized dialysis devices that could enhance in-home low flow rate nocturnal dialysis.

Stage 2 Electrodialysis (ED) Recovery of Plasma Salt

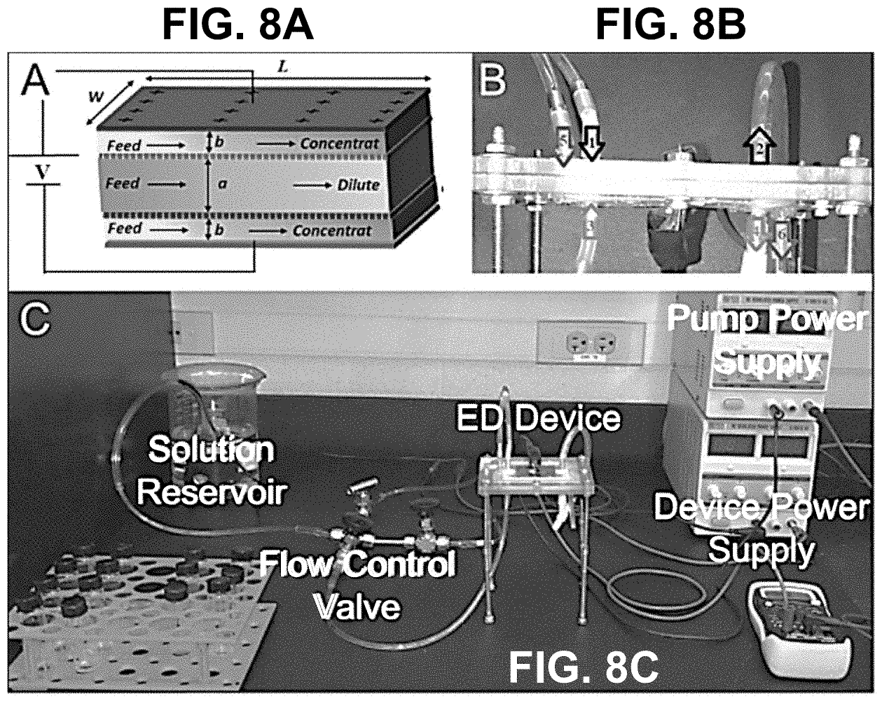

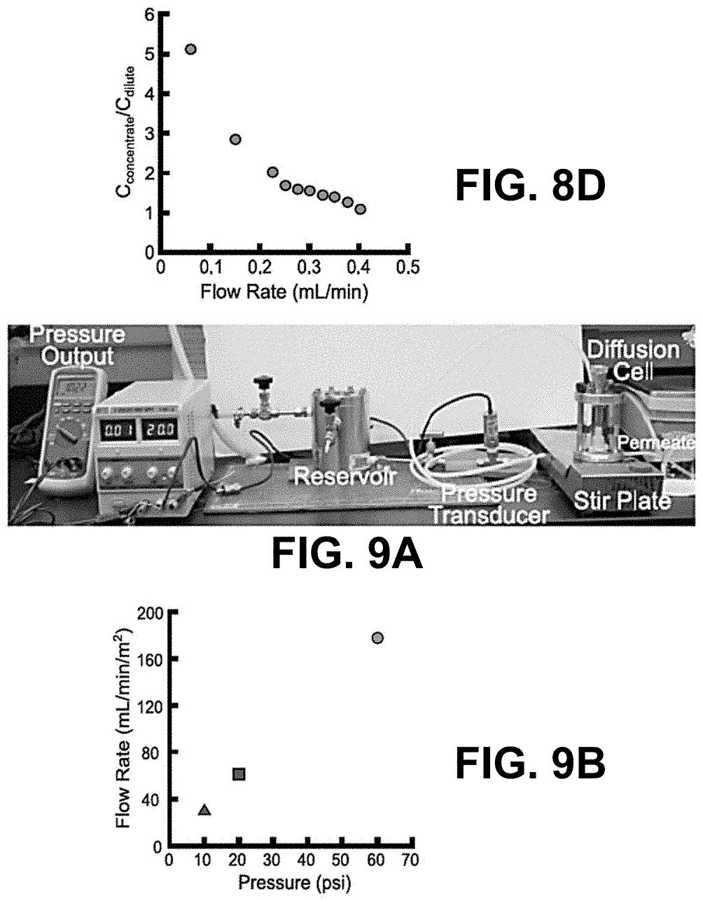

Casting, forming and bonding techniques paired with use of CEM and AEM commercial membranes can be used to develop a multilayer ED module (about 5×5 cm²). To demonstrate feasibility of the ED desalting process, an experimental study was conducted. FIGS. 8A-8C illustrate the experimental setup, with two non-optimal CEM and AEM membranes (thickness about 450 μm) being used for initial desalting characterization. FIG. 8A is a schematic diagram visualizing operation of the state 2 (blood filtration) module (503, FIG. 5), and FIGS. 8B and 8C are images of the salt inlet/outlet port configuration and the ED desalting test setup. The latest commercial membranes are 50-μm-thick with an order of magnitude higher conductivity. NaCl solution at a concentration of 100 mEq/L was supplied to the device at 0.06-0.4 mL/min flow rates for a 2 V applied voltage. As shown in FIG. 8D, a concentration ratio of about 5 (as a function of flow rate at 2 V) was achieved at the lowest flow rate, representing about 70% salt recovery, at 11 mA. The lower flow rate provides ions a longer residence time to transfer to the electrode compartments. Given that some salt is rejected in stage 1 (503) and that a significant amount of salt must be removed, this recovery rate can be adequate.

At a flow rate of 0.06 mL/min, 167 layers are used to process 10 mL/min of fluid. However, these membranes can limit current and consequently the rate of ions recovery. Use of highly conductive 50-μm-thick CEM and AEM membranes can improve operation. The central channel thickness can also be reduced from its initial value of 1 mm to 0.4 mm to reduce ion travel distance as well. The combination of reducing the membrane and channel thickness can result in an order of magnitude increase in current density, which can reduce the number of layers to <15. A multilayer (about 5×5 cm²) module can be fabricated using, e.g., polycarbonate (PC) sheets through thermal forming.

Stage 3 Water Recovery and Waste Removal

Casting, forming and bonding techniques paired with RO membranes can be used to fabricate a multilayer RO membrane module (about 5×5 cm²) for recovery of water from the stage 2 device (509) exit stream. The function of this stage is to cleanse water leaving the stage 2 device (509), stream 6 shown in FIG. 5. Water can be cleansed using RO membranes, which can be made of a thin polyamide layer (e.g., <200 nm) atop of a polyethersulfone or polysulfone porous layer (e.g., about 50-μm-thick) over a non-woven fabric support sheet. The three-layer configuration gives the desired properties of high rejection of undesired species (like salts), high filtration rate, and good mechanical strength. The polyamide top layer is responsible for the high rejection and can be chosen primarily for its permeability to water and relative impermeability to various dissolved impurities including salt ions and other small, unfilterable molecules. Urea is the smallest toxin that should be filtered at this stage.

To evaluate efficacy of this filtration stage, a preliminary study was conducted to determine rejection rate of urea. FIG. 9A is an image showing the experimental setup, including a GE Osmonic Suez RO membrane. A 10 mM urea solution was prepared and passed through a deadend cell with gentle stirring at 10, 20, and 60 psi supply pressure produced over the feed liquid using a nitrogen supply line. In other implementations, a pump can be used in the system. The solution was allowed to equilibrate for one hour before permeate collection to sufficiently wet the membrane. Permeate samples were subsequently collected over a 4 hours period. A urea rejection rate of about 80% was observed across the tested pressure conditions. FIG. 9B presents the volumetric flow rate versus applied pressure for each scenario. A linear improvement is water permeation as a function of pressure was observed.

System Assembly and Testing

The three-stage system of FIG. 5 can be integrated. The system can perform CRRT without dialysate or reinfusion fluid. A breadboard assembly of the three stages can be prepared and it performance tested over a wide range of working conditions. A GO nanoplatelet spacing from the 3-5 nm range can be selected based on the rest results and the stage 1 module (503) can be fabricated. An experimental setup comprising the three stages can be assembled. The systems can be instrumented to enable measurement of pressure distribution, flow rates and salt concentration (via measurement of conductance). Tests conducted with water can be used to evaluate flow and pressure distribution within the system. NaCl at a concentration of 140 mEq/L can be supplied to the system (through stage 1) and the effect of stage 2 (FIG. 5) applied voltage on the system exit salt concentration can be measured. Then, a solution of Na⁺, K⁺, Cl⁻, HCO³⁻, Mg²⁺, Ca²⁺, PO₄³⁻, and/or SO₄²⁻ at physiological concentration can be prepared and the capability of the system to recover and remove these salts can be evaluated. Urea, B2M, interleukin-18, adiponectin, and pentraxin-3 can be added to the salt solution and the performance of the entire system in terms of salts and water recovery, waste discharge and power consumption can be studied under different flow rates, applied voltage, and stage 3 pressure. Based on these studies, a set of tests scenarios can be defined for the bovine blood testing.

Whole Blood Clearance

The operating parameters of the three-stage CRRT system (FIG. 5) can be optimized using whole bovine blood. A dialysate-free three-stage CRRT system can maintain the uremic toxin levels better than the existing HD systems. A recirculating 1 L whole blood circuit can be adapted to the three-stage system. Concentrations and flow rates at each stage can be measured continuously. Measurements can be used to confirm that the GO membrane module is hemocompatible. Incorporation of the second and third stages can produce the data and operating parameters for verification of system operation.

The whole bovine blood can be supplemented initially with cytochrome-c as a mimic for middle-weight toxin B2M during optimization studies. Blood can also be supplemented with urea to mimic ESRD patients. Samples downstream of each stage as well as recirculating blood can be tested every 30 minutes for the first 8 hours and then again at 16 and 24 hours. Operating conditions and parameters can be based on the results, including measure salt, urea and/or middle-weight toxin clearance as a function of pressure and flow rate at stream 3 following stage 1 (503, FIG. 5) using conductance, colorimetric and absorption assays. In stage 2 (506) where salts are recovered from the filtrate, flow rates and conditions at streams 4, 5 and 6 (FIG. 5) can be monitored to understand how the whole blood constituents may alter the results.

Changes in applied voltage, membrane area and module geometries can be implemented in order to recover salts to maintain blood osmolarity when recombined with the water recovery in stage 3 (521). In combination with the salt recovery in stage 2 (506), and the results, water recovery from the RO membrane in stage 3 (521) can be targeted that supports maintenance of blood osmolarity, while simultaneously expelling a urine-like waste in stream 8 (FIG. 5). Osmolarity can be maintained to within 5% of starting conditions and pH within 0.2 of initial readings. Control of the closed recirculating blood loop can be used to determine if gases should be supplemented to maintain pH over 24 hours. The concentrations of urea and cytochrome-c (and B2M) in the waste stream can be monitored and compared against recirculating blood concentrations to determine clearance properties. Data from this can be used to determine the operating conditions and potential modifications of device geometries.

Highly permeable nanomembranes have been engineered to miniaturize the membrane module and provide a novel three-stage filtration system that recapitulates many of the separation, transport and re-absorptive properties of the kidney, eliminating the need for sorbents or large volumes of dialysate. This can enhance adoption of in-home frequent intermittent RRT and develop a truly wearable CRRT. Using ionic nanomembranes, proof-of-concept of the 3 stages to perform kidneys functions and produces urine has be provided.

A microfluidic membrane module with a blood volume of <10 mL, enabled by a nanoengineered membrane, operating at a low flow rate of <100 mL/min (about 140 L/day, similar to a healthy kidney) offers many benefits. The extremely small size and low pressure drop of this device can eliminate the extracorporeal blood circuit and allow operation using arterial pressure (eliminating the blood pump), which can greatly enhance the reliability and safety of dialysis. Blood damage is an unavoidable side effect of extracorporeal circulation because blood is circulated outside the body via one or two peristaltic pumps through a circuit that comprises meters of bloodlines, including needles and chambers. This development can greatly enhance adoption of in-home RRT. Depending on its design, the membrane module can be utilized in both hemodialysis (HD) and hemodiafiltration (HDF) treatment modalities. The microfluidic device can be directly attached to the blood access port, without long tubing, working with arterial pressure. A membrane module comprising a blood filtration membrane can be coupled to a blood access port without an extracorporeal blood circuit. This arrangement can eliminate the need for in-home blood work.

The nanoengineered membrane technology and microfluidic platforms offers similar benefits. The proposed three-stage system eliminates the need for 10-25 L of infusion solution used in HDF. Similar to HFR, the infused solution can be produced internally within the system by reprocessing the filtered plasma. This can minimize or eliminate problems of sterility and apyrogenicity known to cause inflammatory responses, particularly in elderly and patients with diabetes and higher risk of cardiovascular problems. The system can provide online nubs for correction of electrolytes, blood water volume, as well as acid-base balance.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A dialysate-free continuous renal replacement system, comprising:
a blood filtration stage comprising a blood filtration membrane configured to provide a filtered fluid by renal filtration of blood passing through the blood filtration stage at arterial pressure.

2. The dialysate-free continuous renal replacement system of claim 1, wherein the blood passes through the blood filtration stage at a flow rate of 100 ml/min or less.

3. The dialysate-free continuous renal replacement system of claim 2, wherein the blood filtration membrane filters out 10 ml/min of blood plasma or less.

4. The dialysate-free continuous renal replacement system of claim 1, wherein the blood filtration membrane is a graphene oxide (GO) bilayer membrane.

5. The dialysate-free continuous renal replacement system of claim 4, wherein the GO bilayer membrane comprises GO platelets having a flake size of or 150 nm or less.

6. The dialysate-free continuous renal replacement system of claim 5, wherein the GO bilayer membrane comprises a support film with a pore size less than the flake size.

7. The dialysate-free continuous renal replacement system of claim 5, wherein the support film is a poly(methyl methacrylate) (PMMA) film.

8. The dialysate-free continuous renal replacement system of claim 4, wherein the GO bilayer membrane comprises 15 layers or less of GO platelets.

9. The dialysate-free continuous renal replacement system of claim 4, wherein the GO bilayer membrane comprises an interlayer spacing of 5 nm or less.

10. The dialysate-free continuous renal replacement system of claim 9, wherein the interlayer spacing is in a range from about 3 nm to about 5 nm.

11. The dialysate-free continuous renal replacement system of claim 4, wherein the GO bilayer membrane has a permeability of greater than 1500 ml/hr·mmHg·m².

12. The dialysate-free continuous renal replacement system of claim 1, wherein a membrane module comprising the blood filtration membrane is coupled to a blood access port without an extracorporeal blood circuit.

13. The dialysate-free continuous renal replacement system of claim 1, comprising:
a salt recovery stage configured to recover ions through separation from the blood filtration stage; and
a water recovery stage configured to separate water from the desalted fluid from the salt recovery stage, where the water is combined with the separated ions and reinfused into the blood after passing through the blood filtration stage.

14. The dialysate-free continuous renal replacement system of claim 13, wherein the salt recover stage comprises a membrane-based electrodialyzer (ED) comprising an anion exchange membrane (AEM) that separates negative ions from the filtered fluid and a cation exchange membrane (CEM) that separates positive ions from the filtered fluid when under the influence of an electric field established between an anode and a cathode.

15. The dialysate-free continuous renal replacement system of claim 13, wherein the desalted fluid comprises urea and middle-weight toxins.

16. The dialysate-free continuous renal replacement system of claim 13, wherein the water recovery stage comprises a reverse osmosis (RO) membrane comprising a polyamide layer disposed on a polyethersulfone or polysulfone porous layer disposed on a non-woven fabric support sheet.

17. The dialysate-free continuous renal replacement system of claim 16, wherein the polyamide layer has a thickness of about 200 nm or less.

18. The dialysate-free continuous renal replacement system of claim 16, wherein the polyethersulfone or polysulfone porous layer has a thickness of about 50 μm.

19. The dialysate-free continuous renal replacement system of claim 13, wherein separation of water from the desalted fluid generates urine at a rate of about 1-2 mL/min.

20. The dialysate-free continuous renal replacement system of claim 19, wherein the urine is discharged to a urostomy bag.
